(19) United States
(12) Patent Application Publication
(10) Pub. No.: US 2013/0122008 A1
(43) Pub. Date: May 16, 2013

(54) Anti-IL 1- ß Antibody Combination Therapy

(71) Applicants: Matthew Goodman, Guildford (GB); Mariadele Noè, Huningue (FR)

(72) Inventors: Matthew Goodman, Guildford (GB); Mariadele Noè, Huningue (FR)

(73) Assignee: NOVARTIS AG

(21) Appl. No.: 13/318,626

(22) PCT Filed: May 5, 2010
(86) PCT No.: PCT/EP10/56133

§ 371 Date: Nov. 3, 2011

Related U.S. Application Data
(60) Provisional application No. 61/175,922, filed on May 6, 2009.

(51) Int. Cl. A61K 45/06; A61K 31/155; A61K 39/395

(52) U.S. Cl. CPC A61K 45/06; A61K 39/3955; A61K 31/155
USPC 424/139.1

(57) ABSTRACT

The present invention relates to a new combination comprising a therapeutically effective amount of an anti-IL1β antibody or an antigen-binding fragment thereof and at least one anti-diabetic agent. The anti-diabetic agent can be selected from the group consisting of insulin signaling pathway modulators, such as inhibitors of protein tyrosine phosphatases (PTPases), non-small molecule mimetic compounds and inhibitors of glutamine-fructose-6-phosphate amidotransferase (GFAT), DPP-IV inhibitors, agents influencing a deregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, for simultaneous, separate or sequential use of the anti-IL-1β antibody or a antigen-binding fragment thereof and the at least one anti-diabetic agent, especially in the prevention, delay of progression or treatment of metabolic conditions mediated by IL1β such as conditions of impaired glucose tolerance (IGT), conditions of impaired fasting plasma glucose, metabolic acidosis, ketosis, arthritis, obesity and osteoporosis, in particular diabetes and specifically type 1 diabetes or type 2 diabetes mellitus. The invention also relates to pharmaceutical composition comprising the combination of the invention and methods of treatment using the combination for the prevention, delay of progression or treatment of metabolic conditions mediated by IL1β such as diabetes or improving the function of beta cell function.

The instant application is a 371 of International Patent Application No. PCT/EP2010/056133, filed May 5, 2010, which claims priority to U.S. Provisional Application No. 61/175,922, filed May 6, 2009, the contents of which are hereby incorporated by reference herein in their entirety.

### BACKGROUND

The present invention relates to a new combination comprising a therapeutically effective amount of an anti-IL1β antibody or an antigen-binding fragment thereof and at least one anti-diabetic agent. The anti-diabetic agent can be selected from the group consisting of insulin signaling pathway modulators, such as inhibitors of protein tyrosine phosphatases (PTPases), non-small molecule mimetic compounds and inhibitors of glutamine-fructose-6-phosphate amidotransferase (GFAT), DPP-IV inhibitors, agents influencing a deregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, for simultaneous, separate or sequential use of the anti-IL-1β antibody or a antigen-binding fragment thereof and the at least one anti-diabetic agent, especially in the prevention, delay of progression or treatment of metabolic conditions mediated by IL1β such as conditions of impaired glucose tolerance (IGT), conditions of impaired fasting plasma glucose, metabolic acidosis, ketosis, arthritis, obesity and osteoporosis, in particular diabetes and specifically type 1 diabetes or type 2 diabetes mellitus. The invention also relates to pharmaceutical composition comprising the combination of the invention and methods of treatment using the combination for the prevention, delay of progression or treatment of metabolic conditions mediated by IL1β such as diabetes or improving the function of beta cell function.

Interleukin 1 (IL1) is a cytokine which is produced by cells of the immune system which acts as a mediator of the acute phase of an inflammatory response. Inappropriate or excessive production of IL1, in particular IL1β, is associated with the pathology of various diseases and disorders, such as septicemia, septic or endotoxic shock, allergies, asthma, ischemia, stroke, rheumatoid arthritis and pre-diabetes or diabetes. With regard to pre-diabetes or diabetes, studies of different ethnic groups have shown that beta cells of the pancreas are a major determinant of oral glucose tolerance in subjects with normal and reduced glucose tolerance and that in all populations the progression from normal to impaired glucose tolerance and subsequently to Type 2 diabetes is associated with declining insulin sensitivity and beta-cell function (Kahn, S. E., 2003, Diabetologia 46(1):3-19).

There is always a need to provide an alternative or an improved therapeutic approach for treating diseases or conditions associated with IL1 and in particular IL1β such as diabetes.

### SUMMARY OF THE INVENTION

The present invention provides in one aspect a combination which comprises a therapeutically effective amount of an anti-IL1β antibody or an antigen-binding fragment thereof and at least one anti-diabetic agent.

The present invention provides in a further aspect a combination which comprises therapeutically effective amount of an anti-IL1β antibody or an antigen-binding fragment thereof and at least one anti-diabetic agent for use in the prevention, delay of progression or treatment of type 2 diabetes mellitus.

The present invention also provides a combination which comprises a therapeutically effective amount of an anti-IL1β antibody or an antigen-binding fragment thereof and at least one anti-diabetic agent for use in improving the function of beta cells of the pancreas.

There is also provided in a further aspect, a pharmaceutical composition which comprises a therapeutically effective amount of an anti-IL1β antibody or an antigen-binding fragment thereof and at least one anti-diabetic agent an anti-IL1β antibody or an antigen-binding fragment thereof and at least one anti-diabetic agent with a pharmaceutically acceptable carrier, excipient or diluent.

In another aspect, the present invention provides a method for the prevention, delay of progression or treatment of type 2 diabetes mellitus or improvement of beta cell function in a patient, the method comprising administering to a patient in need thereof a combination which comprises a therapeutically effective amount of an anti-IL1β antibody or an antigen-binding fragment thereof and at least one anti-diabetic agent.

The present invention also provides in a further aspect a kit which comprises a therapeutically effective amount of an anti-IL1β antibody or an antigen-binding fragment thereof; at least one anti-diabetic agent; and instructions for use of the kit.

### BRIEF DESCRIPTION OF THE FIGURES

FIG. 1A. Dose escalation assessments

FIG. 1B. Linear decreasing trend in dose response

FIG. 2A. OGTT-Glucose

FIG. 2B. OGTT-C-peptide

FIG. 2C. OGTT-Glucagon

FIG. 3A. ACZ885 i.v. single dose: 10 mg/kg

FIG. 3B. Fasting plasma glucose

FIG. 4A. Fasting plasma glucose

FIG. 4B. Peak plasma glucose

FIG. 4C. Hemoglobin A1c

### DETAILED DESCRIPTION OF THE INVENTION

The combination of the present invention comprises a therapeutically effective amount of an anti-IL1β antibody or an antigen-binding fragment thereof and at least one anti-diabetic agent.

The anti-diabetic agent can be selected from the group consisting of insulin signaling pathway modulators like, inhibitors of protein tyrosine phosphatases (PTPases), non-small molecule mimetic compounds and inhibitors of glutamine-fructose-6-phosphate amidotransferase (GFAT), DPP-IV inhibitors, agents influencing a deregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, or the pharmaceutically acceptable salts of such a compound.

Preferably, the anti-diabetic agent is selected from the group consisting of inhibitors of GSK-3, retinoid X receptor (RXR) agonists, agonists of Beta-3 AR, insulin, agonists of UCPs, antidiabetic thiazolidinediones (glitazones), non-glitazone type PPARγ agonists, dual PPARγ/PPARα agonists, antidiabetic vanadium containing compounds, biguanides such as metformin.

The term anti-diabetic agent may also be referred to hereinafter as COMBINATION PARTNER. As used herein, the term “anti-diabetic agent” is meant to cover an agent which is suitable for treating an IL-1 mediated disease or condition such as for example type 1 diabetes or type 2 diabetes Mellitus.

The anti-IL1β antibody or antigen-binding fragment thereof in a combination with the COMBINATION PARTNER, is an antibody or an antigen binding fragment thereof that specifically binds IL1β.

Preferably the antibody or antigen-binding fragment thereof is a human monoclonal antibody or antigen-binding fragment thereof which binds human IL1β.

Preferably, the antibody or antigen-binding fragment thereof possesses at least one of the following properties:
a) binds to IL1β ligand or to IL1β receptor;
b) has a selectivity for IL1β; or
c) binds to human IL1β with Kd of 3×10⁻¹⁰ M or less.
d) inhibits the activation of the IL1 pathway.

Preferably, the antibody or antigen-binding fragment thereof inhibits binding of human IL1β ligand to the IL-1β receptor and has at least one of the following properties:
a) cross-competes with a reference antibody for binding to IL1β ligand or to IL1β receptor;
b) completes with a reference antibody for binding to IL1β ligand or to IL1β receptor;
c) binds to the same epitope of IL1β ligand or to IL1β receptor as a reference antibody;
d) binds to IL1β ligand or to IL1β receptor with substantially the same Kd as a reference antibody;
e) binds IL1β ligand or to IL1β receptor with substantially the same off rate as a reference antibody;
wherein the reference antibody comprises an antigen binding site comprising at least one immunoglobulin heavy chain variable domain (V_{H}) which comprises in sequence hypervariable regions CDR1, CDR2 and CDR3, wherein said CDR1 has the amino acid sequence Val-Tyr-Gly-Met-Asn (SEQ ID NO:5), wherein said CDR2 has the amino acid sequence Ile-Ile-Trp-Tyr-Asp-Gly-Asp-Asn-Gln-Tyr-Tyr-Ala-Asp-Ser-Val-Lys-Gly (SEQ ID NO:6), and wherein said CDR3 has the amino acid sequence Asp-Leu-Arg-Thr-Gly-Pro (SEQ ID NO:7);
and direct equivalents thereof.

Preferably, the antibody or antigen-binding fragment thereof inhibits binding of human IL1β, wherein the antibody or antigen-binding fragment thereof comprises at least one antigen binding site comprising:
a) an immunoglobulin heavy chain variable domain (V_{H}) which comprises in sequence hypervariable regions CDR1, CDR2 and CDR3, wherein said CDR1 has the amino acid sequence Val-Tyr-Gly-Met-Asn (SEQ ID NO:5), wherein said CDR2 has the amino acid sequence Ile-Ile-Trp-Tyr-Asp-Gly-Asp-Asn-Gln-Tyr-Tyr-Ala-Asp-Ser-Val-Lys-Gly (SEQ ID NO:6), and wherein said CDR3 has the amino acid sequence Asp-Leu-Arg-Thr-Gly-Pro (SEQ ID NO:7), and
b) an immunoglobulin light chain variable domain (V_{L}) which comprises in sequence hypervariable regions CDR1′, CDR2′ and CDR3′, wherein said CDR1′ has the amino acid sequence Arg-Ala-Ser-Gln-Ser-Ile-Gly-Ser-Ser-Leu-His (SEQ ID NO:8), wherein said CDR2′ has the amino acid sequence Ala-Ser-Gln-Ser-Phe-Ser (SEQ ID NO:9), and wherein said CDR3′ has the amino acid sequence Gln-Gln-Arg-Ser-Asn-Trp-Met-Phe-Pro (SEQ ID NO:10), and
direct equivalents thereof.

Unless otherwise indicated, any polypeptide chain is herein described as having an amino acid sequence starting at the N-terminal extremity and ending at the C-terminal extremity.

The anti-IL1β antibody or antigen-binding fragment thereof is preferably an antibody as described in WO02/16436, hereby incorporated by reference in its entirety. In particular, all SEQ ID Nos referred to herein relate to the sequences actually disclosed in WO0216436.

When the antibody or antigen-binding fragment thereof comprises both the V_{H} and V_{L} domains, these may be located on the same polypeptide molecule or, preferably, each domain may be on a different chain, the V_{H} domain being part of an immunoglobulin heavy chain or fragment thereof and the V_{L} being part of an immunoglobulin light chain or fragment thereof.

By anti-IL1β antibody or antigen-binding fragment thereof is meant a molecule capable of binding or otherwise interacting or associating with the IL1β antigen either alone or associated with other for example scaffolding type molecules. The binding reaction may be shown by standard methods (qualitative type assays) including, for example, a bioassay for determining the inhibition of IL1β binding to its receptor or any kind of binding assays, with reference to a negative control test in which an antibody of unrelated specificity but of the same isotype, e.g. an anti-CD25 antibody, is used. Advantageously, the binding of the anti-IL1β antibody or antigen-binding fragment thereof to IL1β may be shown in a competitive binding assay.

Examples of anti-IL1β antibody or antigen-binding fragment thereof include antibodies or fragments as produced by B-cells or hybridomas and chimeric antibodies, CDR-grafted or human antibodies or any fragment thereof, e.g. F (ab′) 2 and Fab fragments, as well as single chain or single domain antibodies.

A single chain antibody consists of the variable domains of the heavy and light chains of an antibody covalently bound by a peptide linker usually consisting of from 10 to 30 amino acids, preferably from 15 to 25 amino acids. Therefore, such a structure does not include the constant part of the heavy and light chains and it is believed that the small peptide spacer should be less antigenic than a whole constant part.

By “chimeric antibody” is meant an antibody in which the constant regions of heavy or light chains or both are of human origin while the variable domains of both heavy and light chains are of non-human (e. g. murine) origin or of human origin but derived from a different human antibody.

By “CDR-grafted antibody” is meant an antibody in which the hypervariable regions (CDRs) are derived from a donor antibody, such as a non-human (e.g. murine) antibody or a different human antibody, while all or substantially all the other parts of the immunoglobulin e.g. the constant regions and the highly conserved parts of the variable domains, i. e. the framework regions, are derived from an acceptor antibody, e. g. an antibody of human origin. A CDR-grafted antibody may however contain a few amino acids of the donor sequence in the framework regions, for instance in the parts of the framework regions adjacent to the hypervariable regions.

By “human antibody” is meant an antibody in which the constant and variable regions of both the heavy and light chains are all of human origin, or substantially identical to sequences of human origin, not necessarily from the same antibody and includes antibodies produced by mice in which the murine immunoglobulin variable and constant part genes have been replaced by their human counterparts, e. g. as described in general terms in EP0546073 B1, U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, EP0438474B1 and EP0463151B1.

Thus in preferred chimeric antibodies the variable domains of both heavy and light chains are of human origin, for instance those of the ACZ885 antibody (also referred to as Canakinumab) which are shown in SEQ ID NO:1 and SEQ ID NO:2. The nucleotide sequence encoding the VH of SEQ ID NO:1 is set forth as as SEQ ID NO:3 and the nucleotide sequence encoding the VL of SEQ ID NO:2 is set forth as SEQ ID NO:4.

The term “antigen-binding fragment” as used herein refers to one or more fragments of an antibody that retain the capacity of binding or otherwise interacting or associating with the IL1β antigen either alone or associated with other for example scaffolding type molecules. It has been shown that the antigen-binding function of an antibody can be performed by fragments of the full length antibody. Examples of binding fragments encompassed within the term “antigen-binding fragment” of the antibody include but are note limited to:
i) a Fab fragment, a monovalent fragment consisting of the antigen-binding fragment V_{L}, V_{H}, C_{L}, and C_{H}1 domains;
ii) a F(ab′)2 fragment, a bivalent fragment comprising two Fab fragments linked by a disulphide bridge at the hinge region;
iii) a Fd fragment consisting of the V_{H} and C_{H}1 domains;
iv) a Fv fragment consiciting of the V_{L} and V_{H} domains of a single arm of an Ab;
v) a dAb fragment (Ward et al., 1989, Nature 341;544-546), which consists of a V_{H} domain; and
vi) an isolated complementarity determining region (CDR).

Preferably, the antigen-binding fragment is derived from the ACZ885.

Single chain antibodies are also intended to be encompassed within the term “antigen-binding fragment” of the Ab. Other forms of single chain Abs, such as diabodies are also encompassed. Diabodies are bivalent, by specific Abs in which V_{L} and V_{H} domains are expressed on a single polypeptide chain, but using a linker that is too short to allow for pairing between the two domains on the same chain, thereby forcing the domains to pair with complementary domains of another chain and creating two antigen binding sites (Holliger at al., Proc. Natl. Acad. Sci. USA, 90:6444-6448).

The constant region domains preferably also comprise suitable human constant region domains, for instance as described in “Sequences of Proteins of Immunological Interest”, Kabat E. A. at al, U.S. Department of Health and Human Services, Public Health Service, National Institute of Health Hypervariable regions may be associated with any kind of framework regions, though preferably are of human origin. Suitable framework regions are described in Kabat E. A. et al, ibid. The preferred heavy chain framework is a human heavy chain framework, for instance that of the ACZ885 antibody which is shown in SEQ ID NO:1. It consists in sequence of FR1, FR2, FR3 and FR4 regions. In a similar manner, SEQ ID NO:2 shows the preferred ACZ885 light chain framework which consists, in sequence, of FR1′, FR2′, FR3′ and FR4′ regions.

Accordingly, the invention provides a combination comprising an antibody or antigen-binding fragment thereof which comprises at least one antigen binding site comprising either a first domain having an amino acid sequence substantially identical to that shown in SEQ ID NO:1 starting with the amino acid at position 1 and ending with the amino acid at position 118 or a first domain as described above and a second domain having an amino acid sequence substantially identical to that shown in SEQ ID NO:2, starting with the amino acid at position 1 and ending with the amino acid at position 107.

Monoclonal antibodies raised against a protein naturally found in all humans are typically developed in a non-human system e.g. in mice, and as such are typically non-human proteins. As a direct consequence of this, a xenogenic antibody as produced by a hybridoma, when administered to humans, elicits an undesirable immune response which is predominantly mediated by the constant part of the xenogenic immunoglobulin. This clearly limits the use of such antibodies as they cannot be administered over a prolonged period of time. Therefore it is particularly preferred to use single chain, single domain, chimeric, CDR-grafted, or especially human antibodies which are not likely to elicit a substantial allogenic response when administered to humans.

In view of the foregoing, a more preferred antibody or antigen-binding fragment of the invention is selected from a human anti-IL1β antibody which comprises at least:
a) an immunoglobulin heavy chain or fragment thereof which comprises:
   (i) a variable domain comprising in sequence the hypervariable regions CDR1, CDR2 and CDR3, and
   (ii) the constant part or fragment thereof of a human heavy chain;
where said CDR1 has the amino acid sequence Val-Tyr-Gly-Met-Asn (SEQ ID NO:5), where said CDR2 has the amino acid sequence Ile-Ile Trp-Tyr-Asp-Gly-Asp-Asn-Gln-Tyr-Tyr-Ala-Asp-Ser-Val-Lys-Gly (SEQ ID NO:6), and where said CDR3 has the amino acid sequence Asp-Leu-Arg-Thr-Gly-Pro (SEQ ID NO:7); and
b) an immunoglobulin light chain or fragment thereof which comprises:
   (i) a variable domain comprising in sequence the hypervariable regions and optionally also the CDR1′, CDR2′, and CDR3′hypervariable regions, and
   (ii) the constant part or fragment thereof of a human light chain,
where said CDR1′ has the amino acid sequence Arg-Ala-Ser-Gln-Ser-Ile-Gly-Ser-Ser-Leu-His (SEQ ID NO:8), where said CDR2′ has the amino acid sequence Ala-Ser-Gln-Ser-Phe-Ser (SEQ ID NO:9), and where said CDR3′ has the amino acid sequence His-Gln-Ser-Ser-Ser-Leu-Pro (SEQ ID NO:10);
and direct equivalents thereof.

Alternatively, antibody or antigen-binding fragment of the combination may be selected from a single chain binding molecule which comprises an antigen binding site comprising:
a) a first domain comprising in sequence the hypervariable regions CDR1, CDR2 and CDR3, said hypervariable regions having the amino acid sequences as shown in SEQ ID NO:1,
b) A second domain comprising the hypervariable regions CDR1′, CDR2′and CDR3′said hypervariable regions having the amino acid sequences as shown in SEQ ID NO:2, and
c) a peptide linker which is bound either to the N-terminal extremity of the first domain and to the C-terminal extremity of the second domain or to the C-terminal extremity of the first domain and to the N-terminal extremity of second domain;
and direct equivalents thereof.

As it is well known in the art, minor changes in an amino acid sequence such as deletion, addition or substitution of one, a few or even several amino acids may lead to an allelic form of the original protein which has substantially identical properties.

The anti-IL1β antibody or an antigen-binding fragment thereof can incorporate natural and/or unnatural amino acids. The terms “natural and unnatural amino acids” as used herein refers to both naturally occurring amino acids and other non-proteinogenic α-amino acids commonly utilized by those in the peptide chemistry arts when preparing synthetic analogues or naturally occurring peptides, including D and L forms. Naturally occurring amino acids are glycine, alanine, valine, leucine, isoleucine, serine, methionine, threonine, phenylalanine, tyrosine, tryptophan, cysreine, praline, histidine, aspartic acid, asparagines, glutamic acid, glutamine, arginine, ornithine, lysine, and γ-carboxyglutamic acid. Examples of unnatural α-amino acids include hydroxylysine, citrulline, kynurenine, methionine sulfate, aminoalanine, phenylglycine, vinylalanine and others.

It is contemplated that within the context of the present invention, amino acid sequences are at least 80% homologous to one another if they have at least 80% identical amino acid residues in a like position when the sequence are aligned optimally, gaps or insertions in the amino acid sequences being counted as non-identical residues.

The inhibition of the binding of human IL1β to its receptor may be conveniently tested in various assays including such assays are described in WO 02/16436.

By the term “to the same extent” as referred to herein is meant that the reference and the equivalent molecules exhibit, on a statistical basis, essentially identical IL1β binding inhibition curves in one of the assays referred to above. For example, in IL1β binding molecules of the invention typically have IC₅₀s for the inhibition of the binding of IL1β to its receptor which are within +/−x5 of that of, preferably substantially the same as, the IC₅₀ of the corresponding reference molecule when assayed as described above.

For example, the assay used may be an assay of competitive inhibition of binding of IL1β by soluble IL1 receptors or target epitopes and the anti-IL1β antibody or antigen-binding fragment thereof.

Most preferably, the anti-IL1β antibody which is included in the combination of the present invention comprises at least
a) one heavy chain which comprises a variable domain having an amino acid sequence substantially identical to that shown in SEQ ID NO:1 starting with the amino acid at position 1 and ending with the amino acid at position 118 and the constant part of a human heavy chain; and
b) one light chain which comprises a variable domain having an amino acid sequence substantially identical to that shown in SEQ ID NO:2 starting with the amino acid at position 1 and ending with the amino acid at position 107 and the constant part of a human light chain.

Most preferably, the IL1β binding molecule which is included in the combination of the present invention is ACZ885.

The constant part of a human heavy chain may be of the γ₁, γ₂, γ₃, γ₄, μ, α₁, α₂, δ or ε type, preferably of the γ type, more preferably of the γ₁ type, whereas the constant part of a human light chain may be of the κ or λ type (which includes the λ₁, λ₂ and λ₃ subtypes) but is preferably of the κ type. The amino acid sequences of all these constant parts are given in Kabat et al., ibid.

An anti-IL1β antibody or an antigen binding fragment thereof according to the present invention may be produced by recombinant DNA techniques as e.g. described in WO 02/16436.

It is also within the scope of the present invention that the anti-IL1β antibody or the antigen-binding fragment thereof have binding specificity for the antigenic epitope of human IL1β which includes the loop comprising the Glu 64 residue of mature human IL1β (Residue Glu 64 of mature human IL1β correspond to residue 180 of the human IL1 beta precursor). This epitope is outside the recognition site of the IL1beta receptor and it is therefore unexpected that antibodies to this eptitope e.g. the ACZ 885 antibody, are capable of inhibiting the binding of human IL1β to its receptor. Thus the use of such antibodies or antigen-binding fragments there for the treatment of diabetes and in particular type 1 or type 2 diabetes Mellitus is included in the combination of the present invention.

Thus in a further embodiment the invention includes a combination comprising an anti-IL1β antibody or an antigen-binding fragment thereof which has antigen binding specificity for an antigenic epitope of human IL1β which includes the loop comprising residue Glu 64 of mature human IL1β and which is capable of inhibiting the binding of IL-1β to its receptor for the treatment of diabetes and in particular type 1 or type 2 diabetes Mellitus is included in the combination of the present invention.

In yet further embodiment the invention contemplates a combination comprising anti-IL1β antibody or an antigen-binding fragment thereof and at least one anti-diabetic agent comprising:
i) an antibody or an antigen-binding fragment thereof having binding specificity for an antigenic epitope of mature human IL1β which includes the loop comprising Glu 64 and which is capable of inhibiting the binding of IL1β to its receptor, for the prevention and/or treatment of diabetes;
ii) a method for the prevention and/or treatment of diabetes in a patient which comprises administering to the patient an effective amount of an antibody or an antigen-binding fragment thereof having binding specificity for an antigenic epitope of mature human IL1β which includes the loop comprising Glu 64 and which is capable of inhibiting the binding of IL1β to its receptor;
iii) a pharmaceutical composition comprising the an antibody or an antigen-binding fragment thereof having binding specificity for an antigenic epitope of mature human IL1β which includes the loop comprising Glu 64 and which is capable of inhibiting the binding of human IL1β to its receptor, in combination with a pharmaceutically acceptable excipient, diluent or carrier; for the treatment of diabetes in a patient.

For the purposes of the present description an antibody is “capable of inhibiting the binding of human IL1β” if the antibody or the antigen-binding fragment thereof is capable of inhibiting the binding of IL1β to its receptor substantially to the same extent as the ACZ 885 antibody, i.e. has a dissociation equilibrium constant (K_{D}) measured e.g. in a standard BIAcore analysis as disclosed in the Example of 10 nM or lower, e.g. 1 nM or lower, preferably 100 pM or lower, more preferably 50 pM or lower, more preferably 40 pM or lower, more preferably 30 pM or lower, more preferably about 10 pM.

Thus in a yet further aspect the invention provides the use of an antibody to IL1β or an antigen-binding fragment thereof which has a K_{D} for binding to IL1β of about 10 nM, 1 nM, preferably 100 pM, more preferably 50 pM or less more preferably 40 pM or lower, more preferably 30 pM or lower, more preferably about 10 pM for the treatment of diabetes and in particular type 2 diabetes Mellitus. This aspect of the invention also includes uses, methods and compositions for such high affinity antibodies or antigen-binding fragments thereof, as described above binding specificity for an antigenic determinant of mature human IL1β which includes the loop comprising Glu 64.

The anti-IL1β antibody or an antigen-binding fragment thereof can incorporate conservative modifications. As used herein, the term “conservative modification” is intended to refer to amino acid modifications that do not significantly affect or alter the binding characteristics of the anti-IL1β antibody or a antigen-binding fragment thereof containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody or an antigen-binding fragment thereof of the disclosure can be replaced with other amino acid residues from the same side chain family and the altered antibody or an antigen-binding fragment thereof can be tested for retained function. Assays for testing retained function are know to those of skill in the art. In various cases, the heavy and light chains of the anti-IL1β antibody or a antigen-binding fragment thereof may optionally include a signal sequence.

According to one of the aspects of the present invention there is provided a method for the prevention, delay of progression or treatment of IL1 mediated diseases and particularly type 1 and/or 2 diabetes Mellitus or improvement of beta cell function in a patient, the method comprising administering to a patient in need thereof a therapeutically effective amount of a combination which comprises an anti-IL1 β antibody or an antigen-binding fragment thereof and at least one anti-diabetic agent.

In the present application the term “IL1 mediated disease” encompasses all diseases and medical conditions in which IL1 plays a role, whether directly or indirectly, in the disease or medical condition, including the causation, development, progress, persistence or pathology of the disease or condition. Such diseases include septicemia, septic or endotoxic shock, allergies, asthma, bone loss, ischemia, stroke, rheumatoid arthritis and diabetes such as type 1 diabetes or type 2 diabetes Mellitus.

According to one preferred embodiment of the present method, the IL1 mediated disease is type 1 diabetes and/or type 2 diabetes Mellitus.

In the present description the terms “treatment” or “treat” refer to both prophylactic or preventative treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse.

The anti-IL1β antibody or antigen-binding fragment thereof component of the combination is conveniently administered parenterally, intravenously, e.g. into the antercubital or other peripheral vein, intramuscularly, or subcutaneously. Preferably, the anti-IL1β antibody or antigen-binding fragment thereof is administered intravenously. A treatment typically comprises administering the antibody of the invention or an antigen-binding fragment thereof once per every 2 to 3 months, preferably once a month, preferably less frequently, such as once a week. It is envisaged that the treatment regiment may be determine, modified or otherwise altered by the medical practitioner depending on case by case basis.

The anti-IL1β antibody or antigen-binding fragment thereof component of the combination of the present invention may be manufactured in a conventional manner.

The anti-IL1β antibody or antigen-binding fragment thereof is preferably provided in a lyophilized form. For immediate administration this component of the combination of the present invention is dissolved in a suitable aqueous carrier, for example sterile water for injection or sterile buffered physiological saline. If it is considered desirable to make up a solution of larger volume for administration by infusion rather as a bolus injection, it is advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution.

The other component of the combination of the present invention is at least one anti-diabetic agent, also as noted above referred to as the COMBINATION PARTNER. The term “anti-diabetic agent” is used herein throughout interchangeably with the term “anti-diabetic compound” should be understood as having the same meaning i.e. a COMBINATION PARTNER which is suitable for treating an IL-1 mediated disease or condition such as for example type 1 diabetes and/or type 2 diabetes Mellitus.

The at least one COMBINATION PARTNER can be selected from the group consisting of insulin signaling pathway modulators, like inhibitors of protein tyrosine phosphatases (PTPases), non-small molecule mimetic compounds and inhibitors of glutamine-fructose-6-phosphate amidotransferase (GFAT), compounds influencing a deregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, or the pharmaceutically acceptable salts of such a compound and optionally at least one pharmaceutically acceptable carrier.

Examples of “inhibitors of PTPase” include, but are not limited to those disclosed in U.S. Pat. No. 6,057,316, U.S. Pat. No. 6,001,867, WO 99/58518, WO 99/58522, WO 99/46268, WO 99/46267, WO 99/46244, WO 99/46237, WO 99/46236, WO 99/15529 and by Poucheret et al., in Mol. Cell Biochem. 1998, 188, 73-80.

Examples of “non-small molecule mimetic compounds” include, but are not limited to those disclosed in Science 1999, 284; 974-97, especially L-783,281, and WO99/58127, especially CLX-901.

Examples of “inhibitors of GFAT” include, but are not limited to those disclosed in Mol. Cell. Endocrinol. 1997,135(1), 67-77.

The term “inhibitors of G6Pase” used herein means a compound or composition which reduces or inhibits hepatic gluconeogenesis by decreasing or inhibiting the activity of G6Pase. Examples of such compounds are disclosed in WO00/14090, WO99/40062, WO98/40385, EP682024 and Diabetes 1998, 47, 1630-1636.

The term “inhibitors of F-1,6-BPase” used herein means a compound or composition which reduces or inhibits hepatic gluconeogenesis by decreasing or inhibiting the activity of F-1,6-BPase. Examples of such compounds are disclosed in WO00/14095, WO99/47549, WO98/39344, WO98/39343 and WO98/39342.

The term “inhibitors of GP” used herein means a compound or composition which reduces or inhibits hepatic glycogenolysis by decreasing or inhibiting the activity of GP. Examples of such compounds are disclosed in EP978279, U.S. Pat. No. 5,998,463, WO99/26659, EP846464, WO97/31901, WO96/39384, WO9639385 and in particular CP-91149 as described in Proc. Natl. Acad Sci USA 1998, 95, 1776-1781.

The term “glucagon receptor antagonists” as used herein relates in particular to the compounds described in WO98/04528, especially BAY27-9955, and those described in Bioorg Med. Chem. Lett 1992, 2, 915-918, especially CP-99,711, J. Med. Chem. 1998, 41, 5150-5157, especially NNC 92-1687, and J. Biol Chem. 1999, 274; 8694-8697, especially L-168,049 and compounds disclosed in U.S. Pat. No. 5,880,139, WO99/01423, U.S. Pat. No. 5,776,954, WO98/22109, WO98/22108, WO98/21957 and WO97/16442.

The term “inhibitors of PEPCK” used herein means a compound or composition which reduces or inhibits hepatic gluconeogenesis by decreasing or inhibiting the activity of PEPCK. Examples of such compounds are disclosed in U.S. Pat. No. 6,030,837 and Mol. Biol. Diabetes 1994, 2, 283-99.

The term “PDHK inhibitors” as used herein means inhibitors of pyruvate dehydrogenase kinase and include, but are not limited to, those compounds disclosed by Aicher et al., in J. Med. Chem. 42 (1999) 2741-2746.

The term “insulin sensitivity enhancer” used herein means any and all pharmacological active compounds that enhance the tissue sensitivity towards insulin. Insulin sensitivity enhancers include, e.g., inhibitors of GSK-3, retinoid X receptor (RXR) agonists, agonists of Beta-3 AR, agonists of UCPs, antidiabetic thiazolidinediones (glitazones), non-glitazone type PPARγ agonists, dual PPARγ/PPARα agonists, antidiabetic vanadium containing compounds and biguanides such as metformin.

Biguanides, such as metformin are commonly administered in dosage forms containing 500 mg, 750 mg, 850 mg, 1000 mg and 2000 mg or more. Preferably metformin is administered in a dosage of 1000 mg/daily or more.

The insulin sensitivity enhancer is preferably selected from the group consisting of anti-diabetic thiazolidinediones, anti-diabetic vanadium containing compounds and the biguanide metformin.

In one preferred embodiment, the insulin sensitivity enhancer is metformin.

The preparation of metformin (dimethyldiguanide) and its hydrochloride salt is state of the art and was disclosed first by Emil A. Werner and James Bell, J. Chem. Soc. 121, 1922, 1790-1794. Metformin, can be used e.g. in the form as marketed under the trademarks GLUCOPHAGE™.

Examples of “inhibitors of GSK-3” include, but are not limited to those disclosed in WO00/21927 and WO97/41854.

By “RXR agonist” is meant a compound or composition which when combined with RXR homodimers or heterodimers increases the transcriptional regulation activity of RXR, as measured by an assay known to one skilled in the art, including, but not limited to, the “co-transfection” or “cis-trans” assays described or disclosed in U.S. Pat. Nos. 4,981,784, 5,071,773, 5,298,429, 5,506,102, WO89/05355, WO91/06677, WO92/05447, WO93/11235, WO95/18380, PCT/US93/04399, PCT/US94/03795 and CA 2,034,220, which are incorporated by reference herein. It includes, but is not limited to, compounds that preferentially activate RXR over RAR (i.e. RXR specific agonists), and compounds that activate both RXR and RAR (i.e. pan agonists). It also includes compounds that activate RXR in a certain cellular context but not others (i.e. partial agonists). Compounds disclosed or described in the following articles, patents and patent applications which have RXR agonist activity are incorporated by reference herein: U.S. Pat. Nos. 5,399,586 and 5,466,861, WO96/05165, PCT/US95/16842, PCT/US95/16695, PCT/US93/10094, WO94/15901, PCT/US92/11214, WO93/11755, PCT/US93/10166, PCT/US93/10204, WO94/15902, PCT/US93/03944, WO93/21146, provisional applications 60,004,897 and 60,009,884, Boehm, et al., J. Med. Chem. 38(16):3146-3155, 1994, Boehm, at al., J. Med. Chem. 37(18):2930-2941, 1994, Antras et al., J. Biol. Chem. 266:1157-1161 (1991), Salazar-Olivo at al., Biochem. Biophys. Res. Commun. 204:157-263 (1994) and Safanova, Mol. Cell. Endocrin. 104:201-211 (1994). RXR specific agonists include, but are not limited to, LG 100268 (i.e. 2-[1-(3,5,5,8,8-pentamethyl-5,6,7,8-tetrahydro-2-naphthyl)-cyclopropyl]-pyridine-5-carboxylic acid) and LGD 1069 (i.e. 4-[(3,5,5,8,8-pentamethyl-5,6,7,8-tetrahydro-2-naphthyl)-2-carbonyl]-benzoic acid), and analogs, derivatives and pharmaceutically acceptable salts thereof. The structures and syntheses of LG 100268 and LGD 1069 are disclosed in Boehm, at al., J. Med. Chem. 38(16):3146-3155, 1994, incorporated by reference herein. Pan agonists include, but are not limited to, ALRT 1057 (i.e. 9-cis retinoic acid), and analogs, derivatives and pharmaceutically acceptable salts thereof.

Examples of “agonists of Beta-3 AR” include, but are not limited to CL-316,243 (Lederle Laboratories) and those disclosed in WO99/29672, WO 98/32753, WO98/20005, WO98/09625, WO97/46556, WO97/37646 and U.S. Pat. No. 5,705,515.

The term “agonists of UCPs” used herein means agonists of UCP-1, UCP-2 and UCP-3. UCPs are disclosed in Vidal-Puig et al., Biochem. Biophys. Res. Commun., Vol. 235(1) pp. 79-82 (1997). Such agonists are a compound or composition which increases the activity of UCPs.

The antidiabetic thiazolidinedione (glitazone) is, for example, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indol)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)-methyl]phenyl}methane (YM268), 5-{4-[2-(5-methyl-2-phenyl-4-oxazolyl)-2-hydroxyethoxy]-benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenylmethyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7, 8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297).

Preferably, the antidiabetic thiazolidinedione is a compound of formula I,

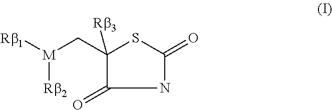

wherein
M represents
   naphthyl, benzoxazolyl, dihydrobenzopyranyl, indole, phenyl (optionally substituted by halogen) or phenylethynyl (optionally substituted by halogen);
Rβ₁ represents halogen or a radical-QRβ₄, in which Q can be oxygen, lower alkylen, carbonyl or —NH—, Rβ₄ is
   naphthyl;
   phenyl, unsubstituted or substituted by 2,4-dioxo-5-thiazolidinyl; or
   lower alkyl or hydroxy lower alkyl, unsubstituted or substituted by
      a) indole or 2,3-dihydroindole,
      b) pyridyl, lower alkyl-pyridyl, N-lower alkyl-N-pyridylamino or halogenphenyl,
      c) dihydrobenzopyranyl, which is unsubstituted or substituted by hydroxy and lower alkyl,
      d) oxazolyl, which is substituted by lower alkyl and phenyl,
      e) cycloalkyl, which is unsubstituted or substituted by lower alkyl, or
      f) arylcycloalkylcarbonyl;
Rβ₂ represents hydrogen or trifluoromethylphenyl-lower alkyl carbamoyl; and
Rβ₃ represents hydrogen or arylsulfonyl;
or a pharmaceutically acceptable salt thereof.

Preferably, the compound of formula VIII is selected from the group consisting of (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297) or a pharmaceutically acceptable salt thereof.

More preferably, the compound of formula VIII is selected from the group consisting of 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone) and 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), MCC555, T-174 and KRP297, especially rosiglitazone, pioglitazone and troglitazone, or a pharmaceutically acceptable salt thereof.

The glitazones 5-{[4-(2-(5-ethyl-2-pyridyl)ethoxy)phenyl]-methyl}thiazolidine-2,4-dione (pioglitazone, EP 0 193 256 A1), 5-{[4-(2-(methyl-2-pyridinyl-amino)-ethoxy)phenyl]methyl}-thiazolidine-2,4-dione (rosiglitazone, EP 0 306 228 A1), 5-{[4-((3,4-dihydro-6-hydroxy-2,5,7,8-tetramethyl-2H-1-benzopyran-2-yl)methoxy)-phenyl]l-methyl}thiazolidine-2 ,4-dione (troglitazone, EP 0 139 421), (S)-((3,4-dihydro-2-(phenyl-methyl)-2H-1-benzopyran-6-yl)methyl-thiazolidine-2,4-dione (englitazone, EP 0 207 605 B1), 5-(2,4-dioxothiazolidin-5-ylmethyl)-2-methoxy-N-(4-trifluoromethyl-benzyl)benzamide (KRP297, JP 10087641-A), 5-[6-(2-fluoro-benzyloxy)naphthalen-2-ylmethyl]thiazolidine-2,4-dione (MCC555, EP 0 604 983 B1), 5-{[4-(3-(5-methyl-2-phenyl-4-oxazolyl)-1-oxopropyl)-phenyl]methyl}-thiazolidine-2,4-dione (darglitazone, EP0332332), 5-(2-naphthylsulfonyl)-thiazolidine-2,4-dione (AY-31637, U.S. Pat. No. 4,997,948), 5-{[4-(1-methyl-cyclohexyl)methoxy)-phenyl]methyl}-thiazolidine-2,4-dione (ciglitazone, U.S. Pat. No. 4,287,200) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The preparation of DRF2189 and of 5-{[4-(2-(2,3-dihydroindol-1-yl)ethoxy)phenyl]methyl}-thiazolidine-2,4-dione is described in B. B. Lohray at al., J. Med. Chem. 1998, 41, 1619-1630; Examples 2d and 3g on pages 1627 and 1628. The preparation of 5-[3-(4-chlorophenyl])-2-propynyl]-5-phenylsulfonyl)-thiazolidine-2,4-dione and the other compounds in which A is phenylethynyl mentioned herein can be carried out according to the methods described in J. Wrobel at al., J. Med. Chem. 1998, 41, 1084-1091.

In particular, MCC555 can be formulated as disclosed on page 49, lines 30 to 45, of EP0604983B1; englitazone as disclosed from page 6, line 52, to page 7, line 6, or analogous to Examples 27 or 28 on page 24 of EP0207605 B1; and darglitazone and 5-{4-[2-(5-methyl-2-phenyl-4-oxazolyl)-ethoxy)]benzyl}-thiazolidine-2,4-dione (BM-13.1246) can be formulated as disclosed on page 8, line 42 to line 54 of EP0332332B1. AY-31637 can be administered as disclosed in column 4, lines 32 to 51 of U.S. Pat. No. 4,997,948 and rosiglitazone as disclosed on page 9, lines 32 to 40 of EP0306228 A1, the latter preferably as its maleate salt. Rosiglitazone can be administered in the form as it is marketed e.g. under the trademark AVANDIA™. Troglitazone can be administered in the form as it is marketed e.g. under the trademarks ReZulin™, PRELAY™, ROMOZIN™ (in the United Kingdom) or NOSCAL™ (in Japan). Pioglitazone can be administered as disclosed in Example 2 of EP 0 193 256 A1, preferably in the form of the monohydrochloride salt. Corresponding to the needs of the single patient it can be possible to administer pioglitazone in the form as it is marketed e.g. under the trademark ACTOS™. Ciglitazone can, for example, be formulated as disclosed in Example 13 of U.S. Pat. No. 4,287,200.

Non-glitazone type PPARγ agonists are especially N-(2-benzoylphenyl)-L-tyrosine analogues, e.g. GI-262570, and JTT501.

The term “dual PPARγ/PPARα agonists” as used herein means compounds which are at the same time PPARγ and PPARα agonists. Preferred dual PPARγ/PPARα agonists are especially those ω-[(oxoquinazolinylalkoxy)phenyl]alkanoates and analogs thereof, very especially the compound DRF-554158, described in WO 99/08501 and the compound NC-2100 described by Fukui in Diabetes 2000, 49(5), 759-767.

Preferably, the antidiabetic vanadium containing compound is a physiologically tolerable vanadium complex of a bidentate monoprotic chelant, wherein said chelant is an α-hydroxypyrone or α-hydroxypyridinone, especially those disclosed in the Examples of U.S. Pat. No. 5,866,563, of which the working examples are hereby incorporated by reference, or a pharmaceutically acceptable salt thereof.

Insulin secretion enhancers are pharmacological active compounds having the property to promote secretion of insulin from pancreatic β cells. Examples for insulin secretion enhancers include glucagon receptor antagonists (see above), sulphonyl urea derivatives, incretin hormones, especially glucagon-like peptide-1 (GLP-1) or GLP-1 agonists, β-cell imidazoline receptor antagonists, and short-acting insulin secretagogues, like antidiabetic phenylacetic acid derivatives, antidiabetic D-phenylalanine derivatives and BTS 67582 described by T. Page et al., in Br. J. Pharmacol. 1997, 122, 1464-1468.

The sulphonyl urea derivative is, for example, glisoxepid, glyburide, glibenclamide, acetohexamide, chloropropamide, glibornuride, tolbutamide, tolazamide, glipizide, carbutamide, gliquidone, glyhexamide, phenbutamide or tolcyclamide; and preferably glimepiride or gliclazide. Tolbutamide, glibenclamide, gliclazide, glibornuride, gliquidone, glisoxepid and glimepiride can be administered e.g. in the form as they are marketed under the trademarks RASTINON HOECHST™, AZUGLUCON™, DIAMICRON™, GLUBORID™, GLURENORM™, PRO-DIABAN™ and AMARYL™, respectively.

GLP-1 is an insulinotropic protein which was described, e.g., by W. E. Schmidt et al. in Diabetologia 28, 1985, 704-707 and in U.S. Pat. No. 5,705,483. The term “GLP-1 agonists” used herein means variants and analogs of GLP-1(7-36)NH₂ which are disclosed in particular in U.S. Pat. No. 5,120,712, U.S. Pat. No. 5,118666, U.S. Pat. No. 5,512,549, WO 91/11457 and by C. Orskov et at in J. Biol. Chem. 264 (1989) 12826. The term “GLP-1 agonists” comprises especially compounds like GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig et al., in Diabetologia 1999, 42, 45-50.

The term “β-cell imidazoline receptor antagonists” as used herein means compounds as those described in WO 00/78726 and by Wang et at in J. Pharmacol. Exp. Ther. 1996; 278; 82-89, e.g. PMS 812.

The anti-diabetic phenylacetic acid derivative is preferably a compound of formula II

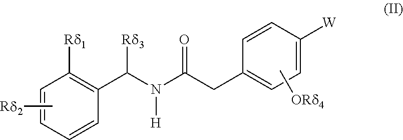

wherein
Rδ₁ is an unbranched C₄-C₆alkyleneimino group which is unsubstituted or mono- or disubstituted by C₁-C₃alkyl;
Rδ₂ is hydrogen, halogen, methyl or methoxy;
Rδ₃ is hydrogen, C₁-C₇alkyl, or phenyl which is unsubstituted or substituted by halogen, methyl or methoxy;
Rδ₄ is hydrogen, allyl, acetyl or propionyl or C₁-C₃alkyl which is unsubstituted or substituted by phenyl; and
W is methyl, hydroxymethyl, formyl, carboxy; or alkoxycarbonyl which comprises between 2 and up to and including 5 carbon atoms and in which the alkyl moiety of the alkoxy group is unsubstituted or substituted by phenyl or a pharmaceutically acceptable salt thereof.

Most preferably, the compound of formula II is repaglinide or a pharmaceutically acceptable salt thereof.

The antidiabetic D-phenylalanine derivative is preferably a compound of formula III

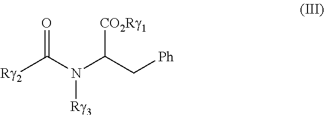

wherein Ph has the meaning of phenyl,
Rγ₁ is selected from hydrogen, C₁ to C₅ alkyl, C₆ to C₁₂ aryl, C₆ to C₁₂ arylalkyl,

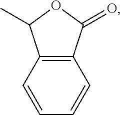

—CH₂CO₂Rγ₃, —CH(CH₃)—OCO—Rγ₃, and —CH₂—OCO—C(CH₃)₃;
Rγ₂ is selected from groups comprising C₆ to C₁₂ aryl, heteroaryl, cycloalkyl, or cycloalkenyl, any of which groups may have one or more substitutents; and
Rγ₃ is selected from hydrogen and C₁ to C₅ alkyl, with the proviso that when Rγ₁ and Rγ₃ are both hydrogen then Rγ₂ is other than substituted or unsubstituted phenyl or naphthyl;
or a pharmaceutically acceptable salts thereof or a precursor which can be converted thereto in the human or animal body.

If Rγ₂ represents heteroaryl, Rγ₂ is preferably quinolynyl, pyridyl or 2-benzofuranyl.

Most preferably, the anti-diabetic D-phenylalanine derivative is nateglinide or a pharmaceutically acceptable salt thereof.

Nateglinide (N-[(trans-4-isopropylcyclohexyl)-carbonyl]-D-phenylalanine, EP196222 and EP526171) and repaglinide ((S)-2-ethoxy-4-{2-[[3-methyl-1-[2-(1-piperidinyl)phenyl]butyl]-amino]-2-oxoethyl}benzoic acid, EP0147850A2, in particular Example 11 on page 61, and EP0207331A1) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The term nateglinide as used herein comprises crystal modifications (polymorphs) such as those disclosed in EP0526171B1 or U.S. Pat. No. 5,488,510, respectively, the subject matter of which is incorporated by reference to this application, especially the subject matter of claims 8 to 10 as well as the corresponding references to the B-type crystal modification. Preferably, in the present invention the B- or H-type, more preferably the H-type, is used. Repaglinide can be administered in the form as it is marketed e.g. under the trademark NovoNorm™. Nateglinide can be used in the form as it is marketed e.g. under the trademark STARLIX™.

α-Glucosidase inhibitors are pharmacological active compounds which inhibit small intestinal α-glucosidase enzymes which break down non-adsorbable complex carbohydrates into absorbable monosaccharides. Examples for such compounds are acarbose, N-(1,3-dihydroxy-2-propyl)valiolamine (voglibose) and the 1-deoxynojirimycin derivative miglitol. Acarbose is 4″,6″-dideoxy-4″-[(1S)-(1,4,6/5)-4,5,6-trihydroxy-3-hydroxymethyl-2-cyclo-hexenylamino}maltotriose. The structure of acarbose can as well be described as O-4,6-dideoxy-4-{[1S,4R,5S,6S]-4,5,6-trihydroxy-3-(hydroxymethyl)-2-cyclohexen-1-yl]amino}-α-D-glucopyranosyl-(1→4)-O-α-D-glucopyranosyl-(1→4)-D-glucopyranose. Acarbose (U.S. Pat. No. 4,062,950 and EP0226121), is generically and specifically disclosed in the documents cited in brackets, in particular in the compound claims and the final products of the working examples, the subject-matter of the final products, the pharmaceutical preparations and the claims are hereby incorporated into the present application by reference to these publications. Corresponding to the needs of the single patient it can be possible to administer acarbose in the form as it is marketed e.g. under the trademark GLUCOBAY™.

Miglitol can be administered in the form as it is marketed e.g. under the trademark DIASTABOL 50™

The α-glucosidase inhibitor is preferably selected from the group consisting of acarbose, voglibose and miglitol.

Examples of “inhibitors of gastric emptying” other than GLP-1 include, but are not limited to those disclosed in J. Clin. Endocrinol. Metab. 2000, 85(3), 1043-1048, especially CCK-8, and in Diabetes Care 1998; 21; 897-893, especially Amylin and analogs thereof, e.g. Pramlintide. Amylin is also described e.g. by O. G. Kolterman et al., in Diabetologia 39, 1996, 492-499.

Examples of “α₂-adrenergic antagonists” include, but are not limited to midaglizole described in Diabetes 36, 1987, 216-220.

Comprised are likewise the corresponding stereoisomers as well as the corresponding polymorphs, e.g. crystal modifications, which are disclosed in the cited patent documents.

In a very preferred embodiment of the invention, the DPP-IV inhibitor is selected from (S)-1-[(3-hydroxy-1-adamantyl)amino]acetyl-2-cyano-pyrrolidine and (S)-1-{2-[5-cyanopyridin-2-yl)amino]ethyl-aminoacetyl}-2-cyano-pyrrolidine, and the further antidiabetic compound is selected from the group consisting of nateglinide, repaglinide, metformin, rosiglitazone, pioglitazone, troglitazone, glisoxepid, glyburide, glibenclamide, acetohexamide, chloro-propamide, glibornuride, tolbutamide, tolazamide, glipizide, carbutamide, gliquidone, glyhexamide, phenbutamide, tolcyclamide, glimepiride and gliclazide, or the pharmaceutically acceptable salt of such a compound.

The term “prevention” means prophylactic administration of the combination to healthy patients to prevent the outbreak of the conditions mentioned herein. Moreover, the term “prevention” means prophylactic administration of such combination to patients being in a pre-stage of the conditions, especially diabetes, to be treated.

The term “delay of progression” used herein means administration of the combination of the present invention comprising an anti-IL1β antibody or a antigen-binding fragment thereof and at least one anti-diabetic agent, to patients being in a pre-stage of a medical condition, particularly diabetes or even more particularly type 2 diabetes Mellitus, to be treated in which patients a pre-form of the corresponding condition is diagnosed.

Examples of the preparation and formulation of inhibitors of PTPases, inhibitors of GSK-3, non-small molecule mimetic compounds, inhibitors of GFAT, inhibitors of G6Pase, glucagon receptor antagonists, inhibitors of PEPCK, inhibitors of F-1,6-BPase, inhibitors of GP, RXR agonists, agonists of Beta-3 AR, PDHK inhibitors, inhibitors of gastric emptying and agonists of UCPs are disclosed in the patents and applications cited beyond each substance listed herein.

The structure of the active agents identified by code Nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The anti-diabetic agents or compounds which are contemplated in the combinations of the present invention can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases. For example, the compounds to be combined can be present as a sodium salt, as a maleate or as a dihydrochloride. The active ingredient or a pharmaceutically acceptable salt thereof may also be used in form of a hydrate or include other solvents used for crystallization.

The COMBINATION PARTNER is preferably selected from the group consisting of insulin signaling pathway modulators, like inhibitors of protein tyrosine phosphatases (PTPases), non-small molecule mimetic compounds and inhibitors of glutamine-fructose-6-phopshate amidotransferase (GFAT), compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, or a pharmaceutically acceptable salt of such a compound.

Preferably, the COMBINATION PARTNER is selected from the group consisting of inhibitors of GSK-3, retinoid X receptor (RXR) agonists, agonists of Beta-3 AR, agonists of UCPs, antidiabetic thiazolidinediones (glitazones), non-glitazone type PPARγ agonists, dual PPARγ/PPARα agonists, antidiabetic vanadium containing compounds, biguanides and metformin.

According to yet another aspect, the present invention relates to a combination comprising an anti-IL1β monoclonal antibody or antigen binding fragment thereof and at least one anti-diabetic agent, wherein the anti-IL1β monoclonal antibody or antigen binding fragment thereof and the at least one anti-diabetic agent are administered simultaneously, separately or sequentially.

The term simultaneous, as used within the context of the present invention means the administration of the at least two components of the combination, by the same route and at the same time or at substantially the same time.

The term separate, as used within the context of the present invention means an administration of the at least two components of the combination at the same time or at substantially the same time by different routes.

The term sequential, as used within the context of the present invention means administration of the at least two components of the combinations at different times, the administration route being identical or different. More particularly by an administration method is meant according to which the whole administration of one of the components of the combination such as the anti-IL1β antibody or a antigen-binding fragment thereof is carried out before administration of the other or others commences. It is thus possible to administer one of the active ingredients or components of the combination over several months before administering the other active ingredient or ingredients. There is no simultaneous treatment in this case. An alternate administration of each active ingredient or component of the combination over several weeks can also be envisaged.

The present invention provides a method for the prevention, delay of progression or treatment of type 2 diabetes mellitus or improvement of beta cell function in a patient, the method comprising administering to a patient in need thereof a therapeutically effective amount of a combination which comprises an anti-IL1β antibody or an antigen-binding fragment thereof and at least one anti-diabetic agent.

According to another aspect the combined preparation which comprises an anti-IL1β antibody or a antigen-binding fragment thereof and at least one further COMBINATION PARTNER and optionally at least one, i.e., one or more, e.g. two, pharmaceutically acceptable carrier for simultaneous, separate or sequential use is in the form of a “kit” or “kit of parts”. It is contemplated that with the kit there is provide label or instructions for use. The label or instructions may provide guidance for use of the anti-IL1β antibody or a antigen-binding fragment thereof and at least one further COMBINATION PARTNER as independent doses or by use of different fixed combinations with distinguished amounts of the components, i.e. at different time points, simultaneously or sequentially. In one embodiment, the label or the instructions of the kit comprise instructions for administration of the antibody or fragment according to any of the dose amounts mentioned herein, numbers of subsequent administrations, and dosing intervals between administrations, as well as any combination of dose amounts numbers of subsequent administrations, and dosing intervals between administrations described herein.

As used herein the term “pharmaceutically acceptable carrier” means any and all solvents, dispersion media, coatings, antibacterial and antifungal agents, isotonic and absorption enhancing or delaying agents and the like that are physiologically compatible. Examples of such pharmaceutically acceptable carriers are water, saline, phosphate buffered saline, acetate buffer with sodium chloride dextrose, glycerol, PEG, ethanol and the like as well as combinations thereof. Additional examples of pharmaceutically acceptable carrier substances are surfactants, wetting agents or minor amounts or auxiliary substances such as wetting or emulsifying agents, preservatives or buffers which enhance the shelf life or effectiveness of the antibody or fragment thereof.

The antibodies of the combination of the present invention can be administered by a variety of methods known in the art, although for many therapeutic applications, the preferred route/mode of administration is subcutaneous, intramuscular, intradermal or intravenous infusion. As will be appreciated by the skilled artisan, the route and/or mode of administration will vary depending upon the desired results. In certain embodiments, the antibody compositions may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York (1978)).

The parts of the kit of parts can then, e.g., be administered simultaneously or chronologically staggered, that is, at different time points and with equal or different time intervals for any part of the kit of parts. Preferably, the time intervals are chosen such that the effect on the treated disease or condition in the combined use of the parts is larger than the effect which would be obtained by use of only any one of the components. In certain embodiments, an anti-IL1β antibody or a antigen-binding fragment thereof of the combination of the invention can be orally administered, for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the anti-IL1β antibody or an antigen-binding fragment thereof can be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The invention also concerns “therapeutically effective amount” or a “prophylactically effective amount” of an antibody or antigen-binding portion of the antibody. A “therapeutically effective amount” refers to an amount effective, at dosages and for periods of time necessary, to achieve the desired therapeutic result. A therapeutically effective amount of the antibody or an antigen-binding portion of the antibody may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

A therapeutically effective amount of each of the components of the combination of the present invention may be administered simultaneously or sequentially or separately. For example, the method of treatment of the invention may comprise (i) administration of the antibody or antigen binding fragment thereof and (ii) administration of at least one further COMBINATION PARTNER simultaneously, sequentially or separately, in jointly therapeutically effective amounts, preferably in synergistically effective amounts, e.g. in daily dosages corresponding to the dosage regiments described herein.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a pre-determined quantity of active compound calculated to produce the desired therapeutic effect in association with the required pharmaceutical carrier. The specification for the dosage unit forms of the invention are dictated by and directly dependent on (a) the unique characteristics of the anti-IL-1β antibody or an antigen-binding fragment thereof and the particular therapeutic or prophylactic effect to be achieved, and (b) the limitations inherent in the art of compounding such an antibody for the treatment of sensitivity in individuals.

The appropriate dosage of the anti-IL1β antibody or the antigen-binding fragment thereof may, of course, vary depending upon, for example, the particular anti-IL1β antibody or a antigen-binding fragment thereof to be employed, the host, the mode of administration and the nature and severity of the condition being treated. However, in prophylactic use, satisfactory results are generally indicated to be obtained at dosages from about 0.05 mg to about 10 mg per kilogram body weight more usually from about 0.1 mg to about 5 mg per kilogram body weight. The frequency of dosing for prophylactic uses will normally be in the range from about once per week up to about once every 3 months, more usually in the range from about once every 2 weeks up to about once every 10 weeks, e. g. once every 4 to 8 weeks.

Further non-limiting ranges for a therapeutically or prophylactically effective amount of an antibody or antigen binding fragment thereof is 0.025 to 50 mg/kg, more preferably 0.1 to 50 mg/kg, more preferably 0.1-25, 0.1 to 10 or 0.1 to 3 mg/kg. In some embodiments, a formulation contains 5 mg/mL of antibody in a buffer of 20 mM sodium acetate, pH 5.5, 140 mM NaCl, and 0.2 mg/mL polysorbate 80. In other embodiments, a formulation contains 10 mg/ml of antibody in 2.73 mg/ml of sodium acetate trihydrate, 45 mg/ml of mannitol, 0.02 mg/ml of disodium EDTA dihydrate, 0.2 mg/ml of polysorbate 80, adjusted to pH 5.5 with glacial acetic acid, e.g. for intravenous use. In other embodiments, a formulation contains 50 mg/ml of antibody, 2.73 mg/ml of sodium acetate trihydrate, 45 mg/ml of mannitol, 0.02 mg/ml of disodium EDTA dihydrate, 0.4 mg/ml of polysorbate 80, adjusted to pH 5.5 with glacial acetic acid, e.g. for subcutaneous or intradermal use. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In a preferred embodiment, the therapeutically or prophylactically effective amount of the antibody or the antigen binding fragment thereof is 0.03 mg/kg.

In a further more preferred embodiment, the therapeutically or prophylactically effective amount of the antibody or the antigen binding fragment thereof is 0.1 mg/kg.

In yet a further more preferred embodiment, the therapeutically or prophylactically effective amount of the antibody or the antigen binding fragment thereof is 0.3 mg/kg.

In yet a further preferred embodiment, the therapeutically or prophylactically effective amount of the antibody or the antigen binding fragment thereof is 1.5 mg/kg.

In yet a further more preferred embodiment, the therapeutically or prophylactically effective amount of the antibody or the antigen binding fragment thereof is 10 mg/kg.

In a preferred embodiment, the therapeutically or prophylactically effective amount of the antibody or the antigen binding fragment thereof is 0.03 mg/kg and the amount of metformin is ≧1000 mg/day.

In a further more preferred embodiment, the therapeutically or prophylactically effective amount of the antibody or the antigen binding fragment thereof is 0.1 mg/kg and the amount of metformin is ≧1000 mg/day.

In yet a further more preferred embodiment, the therapeutically or prophylactically effective amount of the antibody or the antigen binding fragment thereof is 0.3 mg/kg and the amount of metformin is ≧1000 mg/day.

In yet a further preferred embodiment, the therapeutically or prophylactically effective amount of the antibody or the antigen binding fragment thereof is 1.5 mg/kg and the amount of metformin is ≧1000 mg/day.

In yet a further more preferred embodiment, the therapeutically or prophylactically effective amount of the antibody or the antigen binding fragment thereof is 10 mg/kg and the amount of metformin is ≧1000 mg/day.

According to one aspect of the present invention, there is provided a kit or kit of parts comprising the combination of the present invention and instructions for use and optionally dose selection.

The kit can further contain one or more additional reagents, such as an immunosuppressive reagent, a cytotoxic agent or a radiotoxic agent, or one or more additional human antibodies or antigen binding fragments thereof of the disclosure (e.g., a human antibody having a complementary activity which binds to an epitope in the IL1β antigen distinct from the first human antibody) and the least one further COMBINATION PARTNER.

Preferably, there is at least one beneficial effect by the combination of the present invention, e.g. a mutual enhancing of the effect of the anti-IL1β antibody or the antigen-binding fragment thereof and at least one further COMBINATION PARTNER, additional advantageous effects, less side effects, a combined therapeutic effect in a non-effective dosage of one or each of the components of the combination, and especially a synergism, e.g. a more than additive effect, between an anti-IL1β antibody or a antigen-binding fragment thereof and at least one further COMBINATION PARTNER.

Unexpectedly, it was observed that administering a therapeutically effective combination to patients, the combined administration of 1.5 mg/kg of the anti-IL1β antibody and about 1000 mg/daily dose of metformin or more such as 1500 mg/daily or 2000 mg/daily, achieved a statistically significant reduction of hemoglobin A1c (HbA1c) in the group of patients relative to each one of the anti-IL1β antibody or metformin tested alone in a control group of patients. This finding was surprising and unexpected.

It was also unexpectedly observed that patients on a long period of treatment such as over 1 month or more, preferably over 2 months or more, more preferably over 4 months or more, even more preferably over 6 months of treatment or more exhibit maintenance of post-prandial glucose (PPG) which is indicative of an improvement of the beta-cell function of the pancreas in T2DM patients.

According to one of the aspects of the present invention there is provided a combination comprising a therapeutically effective amount of an anti-IL1β antibody or an antigen binding fragment thereof and metformin for use in improving beta-cell function of the pancreas in a patient.

The nature of the conditions mediated by IL1 and particularly IL1β, such as diabetes is multi-factorial. Under certain circumstances, drugs with different mechanisms of action may be combined. However, just considering any combination of drugs having different mode of action but acting in the similar field does not necessarily lead to combinations with advantageous effects.

All the more surprising is the experimental finding that the combined administration of an anti-IL1β antibody or a antigen-binding fragment thereof and at least one further COMBINATION PARTNER results not only in a beneficial, especially a synergistic, therapeutic effect but also in additional benefits resulting from combined treatment such as a surprising prolongation of efficacy, a broader variety of therapeutic treatment, and surprising beneficial effects on diseases and conditions associated with diabetes, e.g. less gain of weight.

The combination of the present invention is also beneficial to patients who display gastrointestinal sides effects as a result of first line anti-diabetic therapy e.g. metformin. Such gastrointestinal side effects can also affect the dose at which metformin is used, if at all, and its efficaciousness. Advantageously, the combination of the present invention can not only lead to a reduction in gastrointestinal side effects associated with metformin but also lead to the toleration of much higher doses of metformin. Thus, leading to a more effective diabetes therapy.

Further benefits are that lower doses of the individual components of the combination such as the anti-IL1β antibody or a antigen-binding fragment thereof and the at least one further COMBINATION PARTNER to be combined according to the present invention can be used in reduced dosage form, for example, that the dosages need not only often be smaller but are also applied less frequently, or can be used in order to diminish the incidence of undesirable side effects. This being recognized as one of the main concerns and a desired requirement of the patients to be treated.

It can be shown by established test models and especially those test models described herein that the combination of an anti-IL1β antibody or a antigen-binding fragment thereof and at least one further COMBINATION PARTNER results in a more effective prevention or preferably treatment of conditions mediated by IL1β, in particular diabetes, especially type 2 diabetes Mellitus.

The person skilled in the pertinent art is fully enabled to select a relevant animal test model to prove the herein throughout indicated therapeutic indications and beneficial effects. The pharmacological activity may, for example, be demonstrated following essentially an in vivo test procedure in mice or in a clinical study as described herein.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The invention is further described by way of illustration only in the following Examples.

### Methodology and Examples

### In-Vivo Test in Mice for Blood Glucose Control

ICR-CDI mice (male, five weeks old, body weight: about 20 g) are abstained from food for 18 hours, and then used as test subjects. The combination according to the present invention and the active ingredients alone are suspended in 0.5% CMC-0.14M sodium chloride buffer solution (pH 7.4). The solution thus obtained is administered orally in fixed volume amounts to the test subjects. After predetermined time, the percentage decrease of the blood glucose against the control group is determined. Further details of the in vitro test are available in Osborn et al., 2008 (hereby incorporated by reference in its entirety), Title: Treatment with Interleukin 1 beta antibody improves glycemic control in diet-induced obesity.

### Hemoglobin A1c

The hemoglobin A1c assay is performed at Covance Central Laboratory Services (CLS) on the FDA-approved Bio-Rad Variant™ analyzer. This analyzer utilizes the principles of ion-exchange high performance liquid chromatography (HPLC) and microcomputer technology. Detection is performed at two wavelengths, 415 nm and 690 nm, to insure a stable baseline. A chromatogram of the changes in the absorbance is plotted versus the retention time. Each chromatogram printout is accompanied by a report identifying each peak detected, plus the relative percent and retention times of each peak.

The method has an inter-assay precision of 1.7-2.1% CV, and an intra-assay precision of 0.9-1.1% (determined by assaying 15 replicate assays of a sample then evaluating the coefficient of variation).

High Sensitive C-Reactive Protein (hsCRP)

CRP is one of the “acute phase” proteins, the serum or protein levels of which rise during general, nonspecific response to infectious and non-infectious inflammatory processes such as rheumatoid arthritis, cardiovascular disease and peripheral vascular disease. CRP is synthesized in the liver and is present in trace amounts in serum or plasma. The C-Reactive Protein HS assay is performed at Covance Central Laboratory Services by immunonephelometry using the Siemens BNII Nephelometer. Polystyrene particles coated with monoclonal antibodies to CRP are agglutinated when mixed with samples containing CRP. The intensity of the scattered light in the nephelometer depends on the CRP content of the sample and therefore the CRP concentration can be determined versus dilutions of a standard of a known concentration

The method has an inter-assay precision of 2.1-5.7% CV, and an intra-assay precision of 2.3-4.4% (determined by assaying 10 replicate assays of a sample then evaluating the coefficient of variation). CRP accuracy is evaluated by comparison with College of American Pathologists (CAP) Cardiac Risk Survey.

### Plasma Glucose

The glucose assay is a hexokinase enzymatic method. Hexokinase catalyzes the phosphorylation of glucose with adenosine triphosphate. The glucose-6-phosphate is then oxidized to 6-phosphogluconate in the presence of NAD by the enzyme glucose-6-phosphate dehydrogenase. The amount of NADPH formed during the reaction is equivalent to the amount of D-glucose in the specimen and is measured photometrically by the increase in absorbance.

The method has an inter-assay precision of 5.1-6.2% CV, and an intra-assay precision of 1.7-2.5% (determined by assaying 50 replicate assays of a sample then evaluating the coefficient of variation). The method is specific for glucose; no other carbohydrate is oxidized.

### C-Peptide

In the beta cells of the pancreas, the proinsulin molecule is cleaved to form insulin and C-Peptide. C-Peptide, a polypeptide consisting of 31 amino acids, is stored in the secretory granules of the beta cells and released into circulation in equimolar amounts with insulin. The determination of C-Peptide provides an assessment of endogenous insulin secretory reserves in patients with diabetes mellitus and is considered a more reliable indicator of insulin secretion than insulin itself.

The ADVIA Centaur C-Peptide assay is a two-site sandwich immunoassay using direct chemiluminescent technology, which uses constant amounts of two antibodies. The first antibody, in the Lite Reagent, is a monoclonal mouse anti-C-Peptide antibody labeled with acridinium ester. The second antibody, in the Solid Phase, is a monoclonal mouse anti-C-Peptide antibody. Streptavidin in the Solid phase is covalently coupled. A direct relationship exists between the amount of C-Peptide present in the patient sample and the amount of relative light units (RLUs) detected by the system.

The method has an inter-assay precision of 1.69-1.81% CV, and an intra-assay precision of 3.7-4.1% (determined by assaying 20 replicate assays of a sample then evaluating the coefficient of variation). Accuracy is evaluated by comparison with College of American Pathologists (CAP) Ligand.

### Glucagon

Glucagon is a hormone secreted by the alpha cells of the pancreatic islets of Langerhans. It is secreted in response to hypoglycemia and increases the blood glucose. As serum glucose levels rise in the blood, glucagon is inhibited by a negative feedback mechanism. The Millipore/LINCO Glucagon Radioimmunoassay kit utilized I¹²⁵ labeled glucagon and glucagon antiserum to determine the level of glucagon in plasma by the double antibody/PEG technique. The antibody is specific for pancreatic glucagon. A standard curve is set up with increasing concentrations of standard unlabeled antigen and from this curve the amount of antigen in unknown samples can be calculated.

The method has an inter-assay precision of 7.3-13.5% CV, and an intra-assay precision of 4.0-6.8% (determined by assaying replicate assays then evaluating the coefficient of variation). Accuracy is evaluated by comparison with College of American Pathologists (CAP) Ligand.

### EXAMPLES

### Example 1

### Clinical Study Design

A multi center, randomized, double blind, placebo-controlled, dose escalation study of the safety, tolerability, pharmacokinetics and pharmacodynamics of ACZ885 administered intravenously to patients with type 2 diabetes mellitus. The aim of the study is to compare the pharmacokinetic and pharmacodynamic effects of five doses (i.v. administration) of ACZ885 with placebo in patients with type 2 diabetes mellitus (T2DM).

Parallel groups of T2DM subjects on a on stable dose of metformin (equal to or higher than 850 mg/day). OGTT was administered at Baseline, 4 and 12 wk post-dose.

### Primary Objective of the Study:

To assess the PD effect of ACZ885 on glycemic indexes in patients with T2DM and glucose response to OGTT

### Secondary Objective(s)

To assess the pharmacodynamic effect of ACZ885 on β cell secretory capacity and insulin sensitivity
To assess the duration of efficacy of a single i.v. dose of ACZ885 (0.03, 0.1, 0.3, 1.5, or 10 mg/kg) on glycemic indexes
To assess the pharmacokinetic effect of ACZ885 in T2DM

Cohort 1 was comprised of 15 patients with T2DM, the adose administered was 0.3 mg/kg ACZ885 or placebo and the main evaluation was safety and tolerability.

The subsequent cohorts were included in the evaluation of pharmacodynamic, pharmacokinetis, pharmacogenomics and pharmacogenetics.
Cohort 2 (n=90; 1:1=ACZ885:Plb): received a single dose of ACZ885 (10 mg/kg) or placebo.
Cohort 3 (n=96) received a single dose of ACZ885 0.1, 0.3, 1.5 mg/kg or placebo (1:1:1:1).
Cohort 4 (n=30; 2:1=ACZ885:P1b) received a single dose of ACZ885 (0.03 mg/kg) or placebo.

### Statistical Analysis:

The study was designed to follow the patients for 24 weeks (168 days) after the single intravenous (i.v.) administration.

An analysis of covariance (ANCOVA) with treatment as the classification variable and baseline as the covariate will be used for the comparison of treatments with respect to these parameters.

### Example 2

### Results of HbA1c and hsCRP Levels

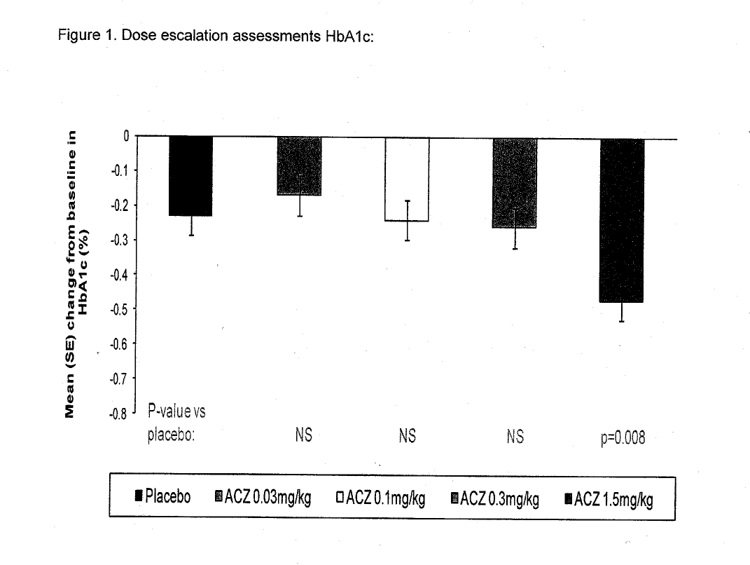

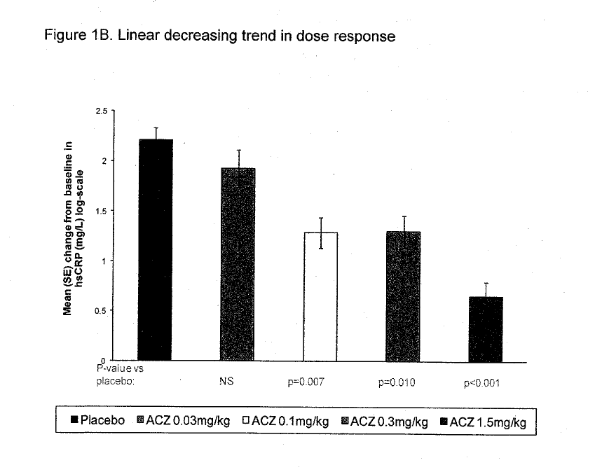

At four weeks after treatment with 0.03, 0.1, 0.3, 1.5 mg/kg ACZ885 in T2DM subjects on a stable dose of metformin resulted in the following:
a statistically significant decreased of hemoglobin A1c (HbA1c) at the dose of 1.5 mg/kg (FIG. 1A),
a linear reduction (dose proportional) in high-sensitivity C-reactive protein (hsCRP) as shown in FIG. 1B.

### Example 3

### Results of Glucose, C-Peptide and Glucagon Levels

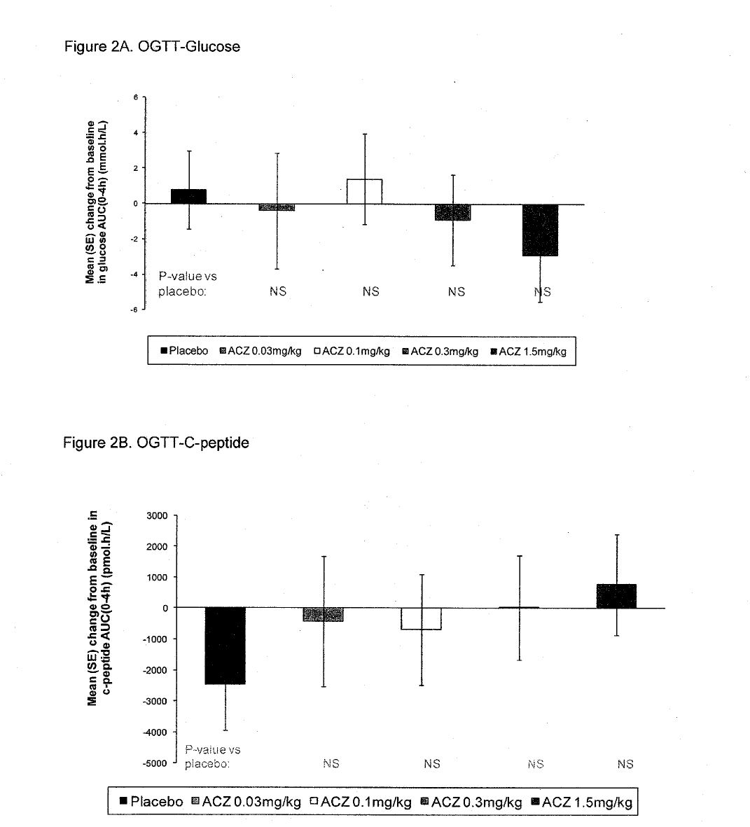

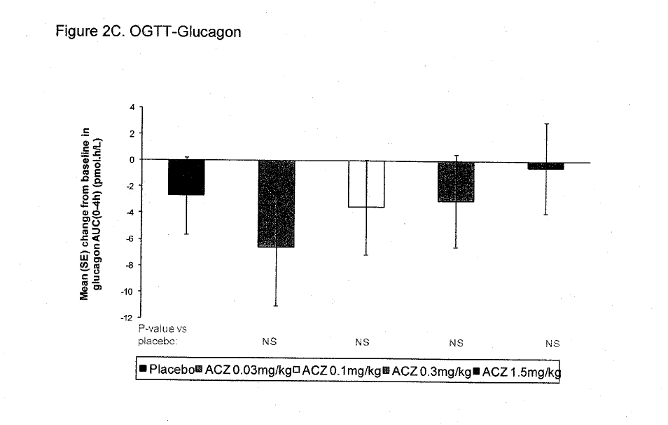

Positive trends consistent with dose escalation were also reported with other OGTT parameters when comparing the area under the curve for the 4 hours interval (AUC0-4 h): glucose (FIG. 2A), C-peptide (FIG. 2B) and glucagon (FIG. 2C).

### Example 4

### Results of Fasting Plasma Glucose

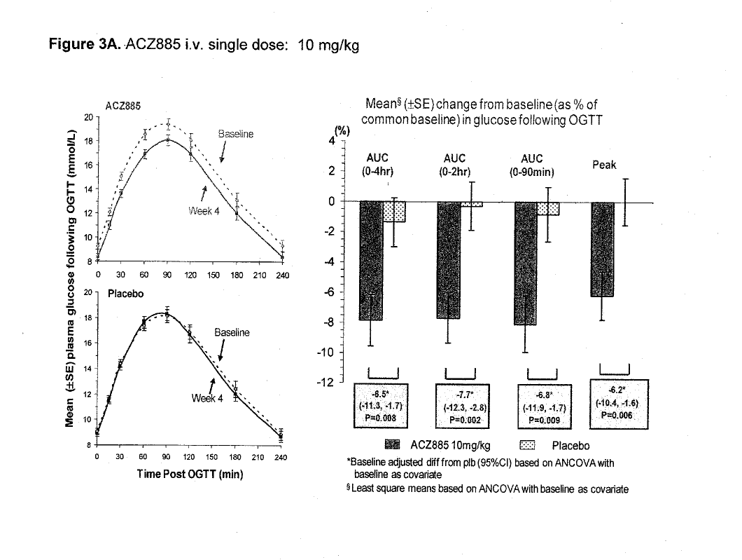

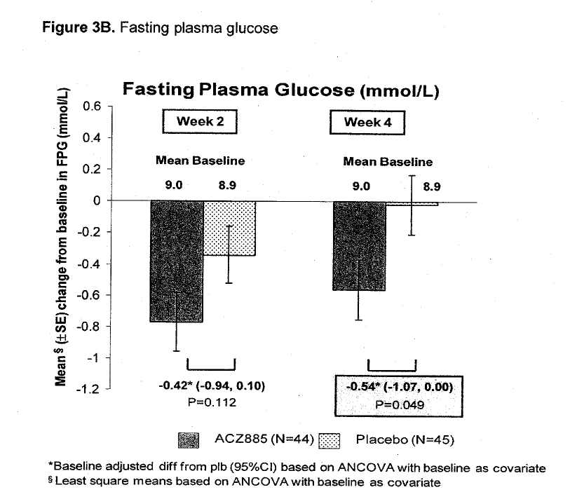

At four weeks after treatment with 10 mg/kg ACZ885 in T2DM subjects on a stable dose of metformin resulted in a statistically significant decreased of plasma AUC glucose following OGTT and a decrease in fasting plasma glucose (FPG) as shown in FIG. 3A.

OGTT Glucose—4 Weeks Following Single Dose of ACZ885 10 mg/kg A significant reduction in postprandial glucose compared to placebo was reported (FIG. 3B).

### Example 5

### Glycemic Parameters

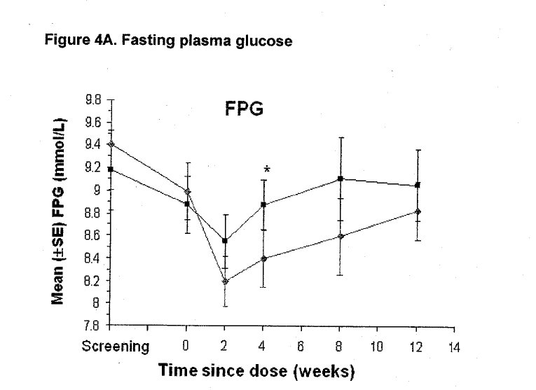

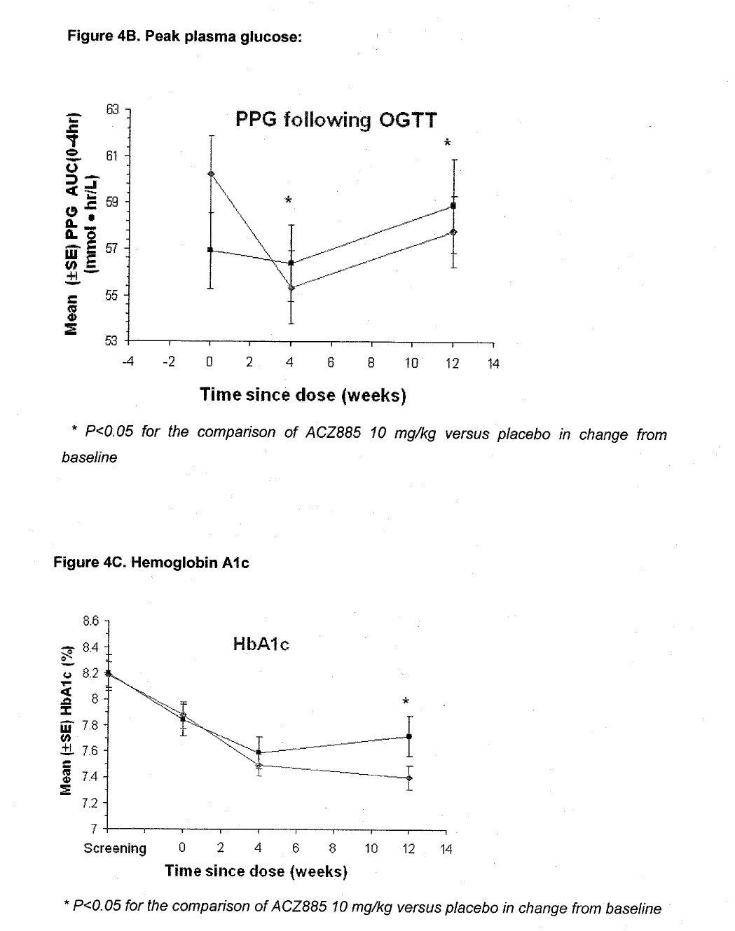

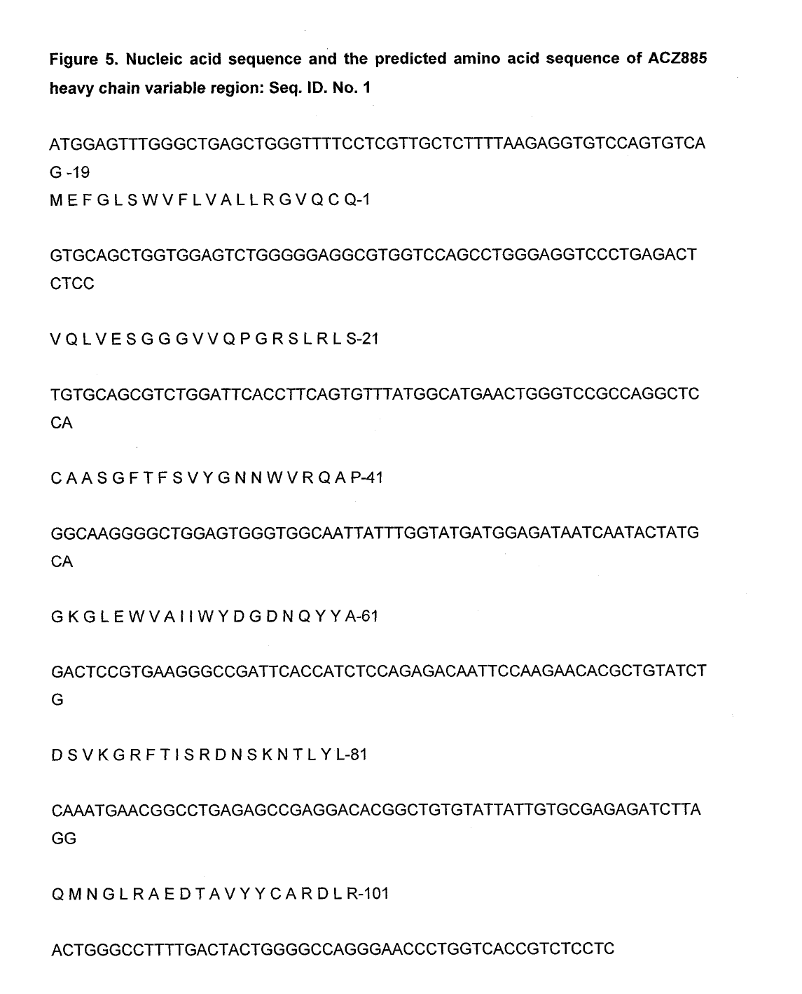

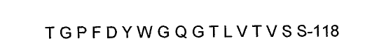

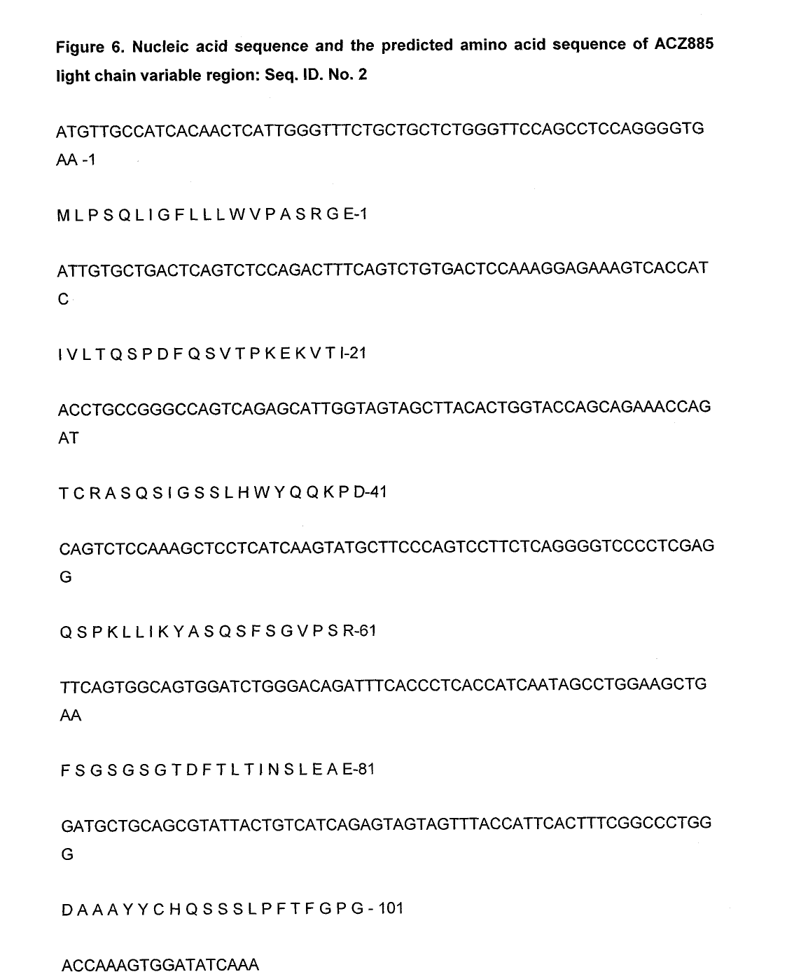

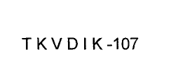

In addition, the time course of pharmacodynamic effects of a single dose of ACZ885 10 mg/kg i.v. on glycemic parameters over a 6 month post-dose are shown in FIGS. 4A, 4B and 4C.

### Fasting Plasma Glucose:

The effect on fasting plasma glucose was statistically significant in the treated group at Week 4 post-dose and attenuated from Week 4 to Week 12 (FIG. 4A).

### Peak Plasma Glucose:

The reduction in peak plasma glucose following OGTT was statistically significant in the treated group at Week 4 and was maintained at Week 12 (FIG. 4B). The symbol (*) denotes. P<0.05 for the comparison of ACZ885 10 mg/kg versus placebo in change from baseline

### Hemoglobin A1c:

In the treated group, a further reduction in HbA1c was observed from Week 4 to Week 12 (−0.32% p=0.032) FIG. 4C. The symbol (*) denotes P<0.05 for the comparison of ACZ885 10 mg/kg versus placebo in change from baseline.

### Conclusions:

Significant reduction in HbA1c was observed for ACZ885 1.5 mg/kg at Week 4. These results demonstrate that a single dose administration of ACZ885 can lead to a statistically significant reduction of HbA1 c when compared to placebo.
Dose response of hsCRP showed a linear decreasing trend at week 4. These results also show a reduction of peak plasma glucose (PPG, following OGTT) at Week 4 and
A positive trend at Week 4 post-dose, albeit not statistically significant, observed in post-prandial glucose, C-peptide, proinsulin, or glucagon versus placebo.
Sustained efficacy following a single dose was reported: A single dose administration of 10 mg/kg ACZ885 elicits a statistically significant reduction of HbA1c versus placebo (−0.32% p=0.032, as well as a reduction of peak plasma glucose (PPG, following OGTT) observed at Week 4 and maintained at Week 12.

Long-term maintenance of PPG indicates an improvement of the beta-cell function in T2DM patients, as shown by the improved peak glucose level excursion following OGTT.

These preliminary data are suggestive of improvement of the beta-cell function in T2DM patients, as shown by the improved HbA1 c, and also supported by the trends in the post-OGTT parameters at 4 weeks post-dose.

1. A pharmaceutical combination comprising a therapeutically effective amount of an anti-IL1β monoclonal antibody or antigen binding fragment thereof and at least one anti-diabetic agent.

2. The combination according to claim 1, wherein the at least one anti-diabetic agent is selected from the group consisting of inhibitors of GSK-3, retinoid X receptor (RXR) agonist, agonist of Beta-3 AR, agonist of UCPs, antidiabetic thiazolidinedione (glitazones), non-glitazone type PPARγ agonist, dual PPARγ/PPARα agonist, antidiabetic vanadium containing compound and a biguanide.

3. The combination according to claim 2, wherein the biguanide is selected from the group consisting of metformin, phenformin and buformin or a pharmaceutically acceptable salt thereof.

4. The combination according to claim 1 wherein the anti-IL1β antibody or antigen-binding fragment thereof is a human monoclonal antibody or antigen-binding fragment thereof.

5. The combination according to claim 4, wherein the human monoclonal anti-IL1β antibody or antigen-binding fragment thereof:
a) binds to IL1β ligand or to IL1β receptor;
b) has a selectivity for IL1β;
c) binds to human IL1β with Kd of 3×10⁻¹⁰ M or less, or
d) inhibits the activation of the IL1 pathway.

6. The combination according to claim 5, wherein the anti-IL1β monoclonal antibody is ACZ885.

7. The combination according to claim 1, wherein the therapeutically effective amount of the anti-IL1β monoclonal antibody is 1.5 mg/kg or 10 mg/kg.

8.-9. (canceled)

10. The combination according to claim 1, wherein the anti-IL1β monoclonal antibody or antigen binding fragment thereof and the at least one anti-diabetic agent are administered simultaneously, separately or sequentially.

11. A pharmaceutical composition comprising the combination according to claim 1 in combination with a pharmaceutically acceptable carrier, excipient or diluent.

12. (canceled)

13. A kit comprising:
i) ACZ885;
ii) metformin; and
iii) instructions for use in the prevention, delay of progression or treatment of type 2 diabetes mellitus or improving beta cell function in a patient in need thereof.

14. A method for the prevention, delay of progression or treatment of type 2 diabetes mellitus or improving beta cell function in a patient in need thereof, comprising administering to the patient a therapeutically effective amount of an anti-IL1β monoclonal antibody or antigen binding fragment thereof and at least one anti-diabetic agent.

15. The method according to claim 14, wherein the at least one anti-diabetic agent is selected from the group consisting of inhibitors of GSK-3, retinoid X receptor (RXR) agonist, agonist of Beta-3 AR, agonist of UCPs, antidiabetic thiazolidinedione (glitazones), non-glitazone type PPARγ agonist, dual PPARγ/PPARα agonist, antidiabetic vanadium containing compound and a biguanide.

16. The method according to claim 15, wherein the biguanide is selected from the group consisting of metformin, phenformin and buformin or a pharmaceutically acceptable salt thereof.

17. The method according to claim 16, wherein the anti-IL1β antibody or antigen-binding fragment thereof is a human monoclonal antibody or antigen-binding fragment thereof.

18. The method combination according to claim 17, wherein the human monoclonal anti-IL1β antibody or antigen-binding fragment thereof:
a) binds to IL1β ligand or to IL1β receptor;
b) has a selectivity for IL1β;
c) binds to human IL1β with Kd of 3×10⁻¹⁰ M or less, or
d) inhibits the activation of the IL1 pathway.

19. The method combination according to claim 18, wherein the anti-IL1β monoclonal antibody is ACZ885.

20. The method combination according to claim 14, wherein the therapeutically effective amount of the anti-IL1β monoclonal antibody is 1.5 mg/kg or 10 mg/kg.

21. The method according to claim 14, wherein the anti-1β monoclonal antibody or antigen binding fragment thereof and the at least one anti-diabetic agent are administered simultaneously, separately or sequentially.


SEQUENCE LISTING (10 sequences)

SEQ ID NO: 1 - protein, 118 aa, homo sapiens [sequence omitted]

SEQ ID NO: 2 - protein, 107 aa, homo sapiens [sequence omitted]

SEQ ID NO: 3 - DNA, 354 bp, Homo sapiens
caggtgcagctggtggagtctgggggaggcgtggtccagcctgggaggtccctgagactctcctgtgcagcgtctggattcaccttcagtgtttatggcatgaactgggtccgccaggctccaggcaaggggctggagtgggtggcaattatttggtatgatggagataatcaatactatgcagactccgtgaagggccgattcaccatctccagagacaattccaagaacacgctgtatctgcaaatgaacggcctgagagccgaggacacggctgtgtattattgtgcgagagatcttaggactgggccttttgactactggggccagggaaccctggtcaccgtctcctct

SEQ ID NO: 4 - DNA, 321 bp, homo sapiens
gaaattgtgctgactcagtctccagactttcagtctgtgactccaaaggagaaagtcaccatcacctgccgggccagtcagagcattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcagcgtattactgtcatcagagtagtagtttaccattcactttcggccctgggaccaaagtggatatcaaa

SEQ ID NO: 5 - protein, 5 aa, HOMO SAPIENS
VYGMN

SEQ ID NO: 6 - protein, 17 aa, HOMO SAPIENS
IIWYDGDNQYYADSVKG

SEQ ID NO: 7 - protein, 6 aa, HOMO SAPIENS
DLRTGP

SEQ ID NO: 8 - protein, 11 aa, HOMO SAPIENS
RASQSIGSSLH

SEQ ID NO: 9 - protein, 6 aa, HOMO SAPIENS
ASQSFS

SEQ ID NO: 10 - protein, 7 aa, HOMO SAPIENS
HQSSSLP